(19) United States
(12) Patent Application Publication
(10) Pub. No.: US 2005/0066403 A1
(43) Pub. Date: Mar. 31, 2005

(54) Eye protection against radiation

(76) Inventor: Walter Kratz, Eitorf (DE)

Correspondence Address: HODGSON RUSS LLP, ONE M & T PLAZA, SUITE 2000, BUFFALO, NY, 14203-2391, US

(21) Appl. No.: 10/868,924

(22) Filed: Jun. 15, 2004

(30) Foreign Application Priority Data
Jun. 16, 2003 (DE) 103 27 017.5

(51) Int. Cl. A61F 9/00

(52) U.S. Cl. 2/15

(57) ABSTRACT

The invention relates to a device for protecting the eyes of a person or user against radiation, comprising at least one arrangement being at least partial little translucent for the radiation for covering one eye or both eyes of the person or user, and an arrangement for attaching the device to the head of a person or user, wherein the device further comprises one or several arrangement(s) allowing a receipt of optical signals from a location being spaced from the device. The invention relates also to a browning apparatus (100) comprising the above device (1). The invention relates also to a method for browning the human body.

The present invention relates to an eye protection against radiation. In particular, the invention relates to a protection device against UV-radiation and/or visible radiation as being radiated from natural or artificial UV-radiation sources, for example from the sun or from tubes of UV-browning apparatuses, for the eyes of a person being exposed to UV-radiation (for example of the sun) or for the eyes of a user of said UV-browning apparatuses.

Browning apparatuses based on UV-radiators are known in various constructions and sizes. Browning apparatuses for whole-body browning comprise a bed (“bench”) as bottom part, if necessary provided with a profile, wherein underneath a surface being transparent for the browning radiation the bed is equipped with UV-radiation sources, for example UV-radiator tubes, radiating the browning radiation. A movable top part corresponds to the bottom part of the browning apparatuses, wherein the top part is pivotable about an axes extending parallel to an edge of the bed, usually parallel to one of those longer edges, or is movable at a support or a suspension at a room ceiling in a vertical direction. Also, above a face being transparent for the browning radiation, the top part is equipped with UV-radiation sources, for example UV-radiator tubes, radiating browning radiation. Users of a browning apparatus lift or pivot the top part in an open position enabling comfortable “entering”, lie down on the bed, close the top part and then switch on the UV-radiator tubes for a pre-selected browning time being within a range of a few minutes, however, possibly being 15 minutes or longer.

Indeed, the radiation radiated from the UV-radiation sources like the UV-radiator tubes is comparatively harmless for the skin, in particular for the skin being accustomed to UV-radiation or for the skin being protected by appropriate protective films (sun creme, sun lotion); however, the user's eyes can be affected by direct contact with intensive UV-radiation already after a short exposition. Therefore, when using UV-browning apparatuses, it is required to wear an appropriate eye protection being non-transparent for the UV-radiation (also for the substantial fraction of the visible radiation) and covering the areas of the eye socket formed by the upper and lower lids.

However, many users infringe upon the instruction applied for their protection and—instead of wearing an eye protection-they simply close the eyes when browning, on the one hand, due to the fact that they feel uncomfortable when wearing the eye protection, on the other hand, for this reason, because when wearing an eye protection while browning said area is not reached by radiation, and consequently, will not be browned, whereby only in the eye region pale areas are formed which are regarded as being unesthetic. In doing so, the danger of a permanent damage of the eyes is not taken into consideration. Therefore, there have been many attempts making wearing of an eye protection obligatory when using a browning apparatus.

One example for it is a device coupling the electric supply of the browning apparatus, and therefore, under switching on the possibility of operating the UV-radiator tubes, with the wearing of an appropriate eye protection. This has been realised in such a manner, that for the apparatus an electrical or electronical operation interlock is switched, either preventing switching on the apparatus when the eye protection is not properly applied, and/or an operation interlock is switching off the apparatus when the user has put off the eye protection during operation.

However, such and similar devices have been side steped by users not willing to wear an eye protection, and do not or only incompletely accomplish their purpose as protection measure.

According to the invention, for solving the problem it is suggested to positively motivate the user of a browning apparatus to wear the eye protection. This is done in such a manner that an arrangement for protecting the eyes against UV-radiation is provided being adapted to receive optical signals, wherein the user of an UV-browning apparatus is interested in receiving the signals. The interest makes the user to wear the eye protection and so—quasi additionally—protects his/her eyes against the UV-radiation and also against most of the fraction of the visible radiation being emitted from the UV-radiator tubes during browning.

Therefore, the invention relates to a device for protecting the eyes of a person or a user against radiation, comprising at least an arrangement being at least partial little translucent for covering one eye or both eyes of the person or user, and an arrangement for attaching the device to the head of the person or user, wherein the device further comprises one or several arrangement(s) allowing receipt of optical signals from a location being spaced from the device.

The invention relates also to a browning apparatus for cosmetic and/or medical applications, comprising:
(a) a static bed having a rest face substantially transparent for browning radiation for the user, and UV-radiation sources emitting browning radiation;
(b) a movable top part having a protection face substantially transparent for browning radiation, and UV-radiation sources emitting browning radiation;
(c) wherein the top part can be opened in relation to the bed for the entering user, and can be closed at the beginning of the browning procedure;
(d) control apparatuses operable by the user for controlling the condition of a browning procedure; and
(e) a device for protection of the eyes of a user against radiation, comprising at least an arrangement being at least partial little translucent for covering an eye or both eyes of the user and an arrangement for attaching the device to the head of the user, wherein the device further comprises:
(f) an or several arrangement(s) allowing a receipt of optical signals from a location spaced from the device.

The invention relates also to a procedure for cosmetic and/or medical browning of at least of parts of the body of a person or the user by means of arrangements for emitting radiation comprising browning radiation under protecting the eyes of the person or user against components of the radiation being harmful for the eyes under simultaneous receipt of optical signals with a device according to following detailed description from a location spaced from the device.

The invention will be further elucidated by the following description with reference to the figures. Preferred examples according to the invention including, such embodiments as being shown in the figures, merely serve for a better demonstration of the invention without limiting the invention thereto.

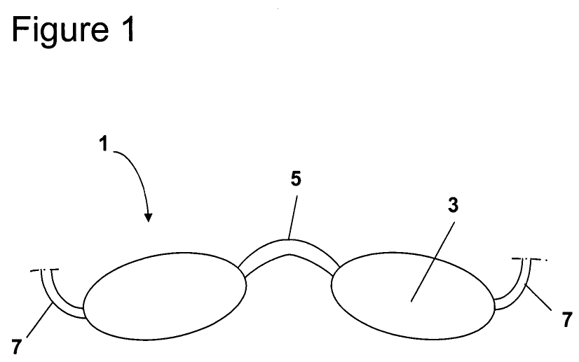

FIG. 1 shows a schematic view of an embodiment of the device 1 according to the invention for covering both eyes of a user.

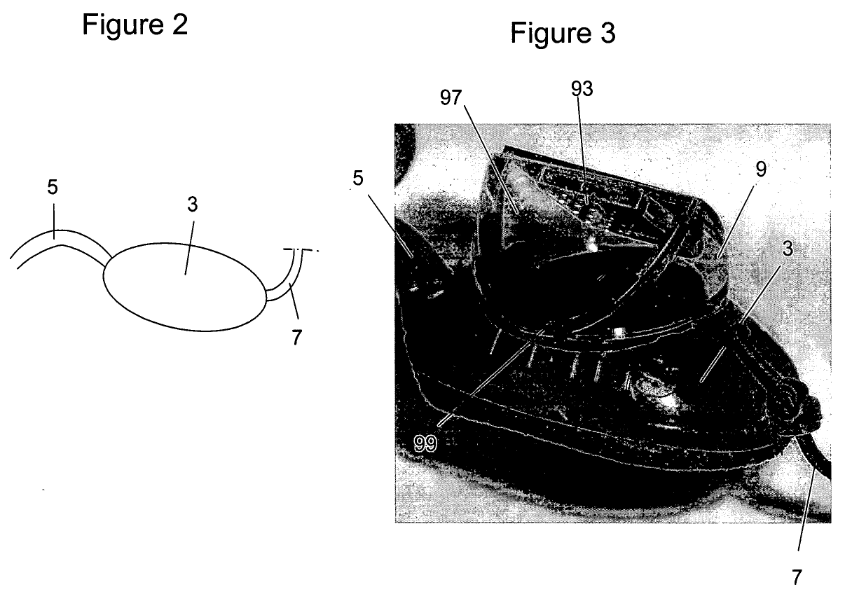

FIG. 2 shows a schematic view of an embodiment of the device 1 according to the invention for covering one eye of a user.

FIG. 3 shows a schematic view of an embodiment of the device 1 according to FIG. 2 with the arrangement for receiving optical signals.

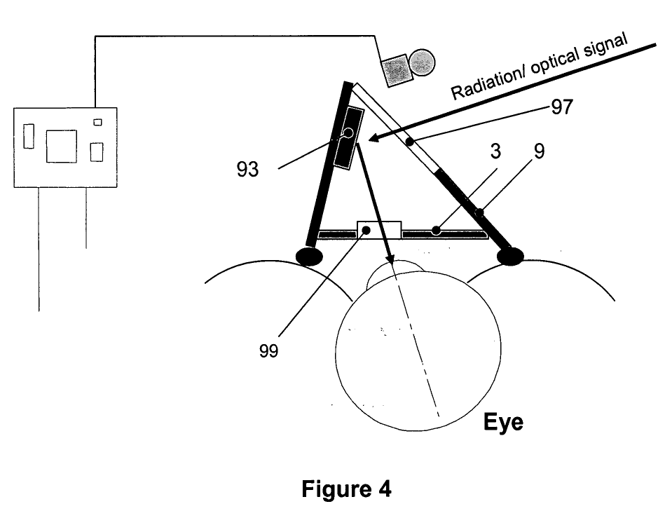

FIG. 4 shows a schematic view of the path of rays in an embodiment of an arrangement according to the invention.

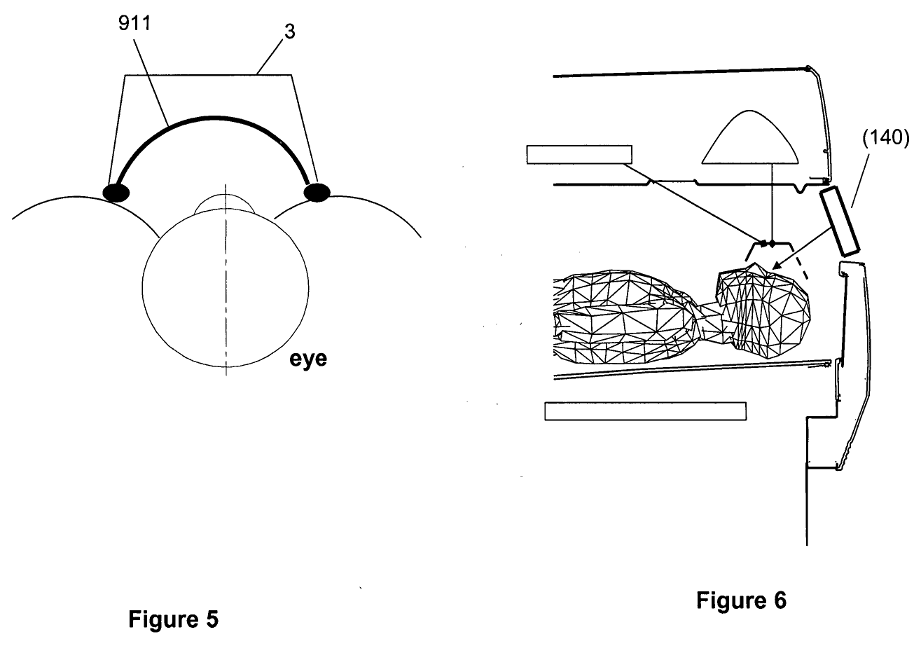

FIG. 5 shows a schematic view of an embodiment of the device according to the invention with a LCD at the inner side of the arrangement for covering the eye.

FIG. 6 shows a portion of a browning bed according to the invention, in which the assembly of the arrangement for emitting optical signals is shown.

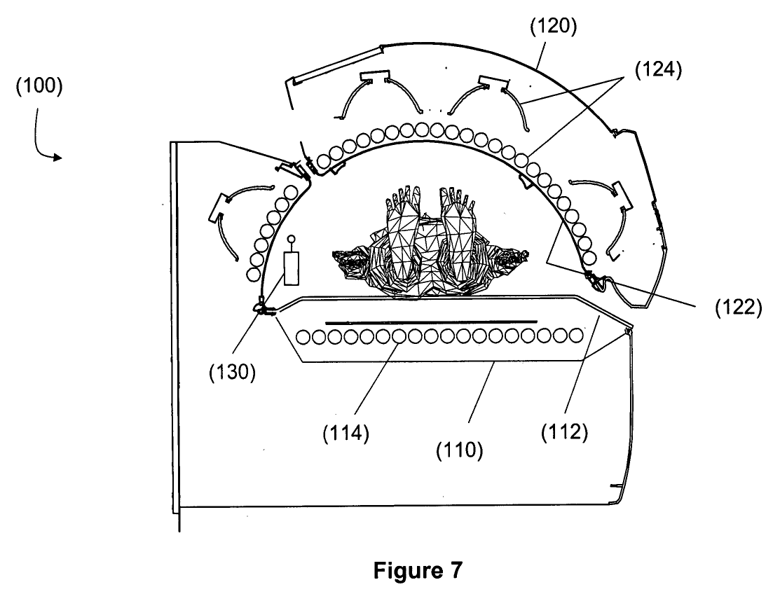

FIG. 7 schematically shows a browning bed according to the invention with various arrangements.

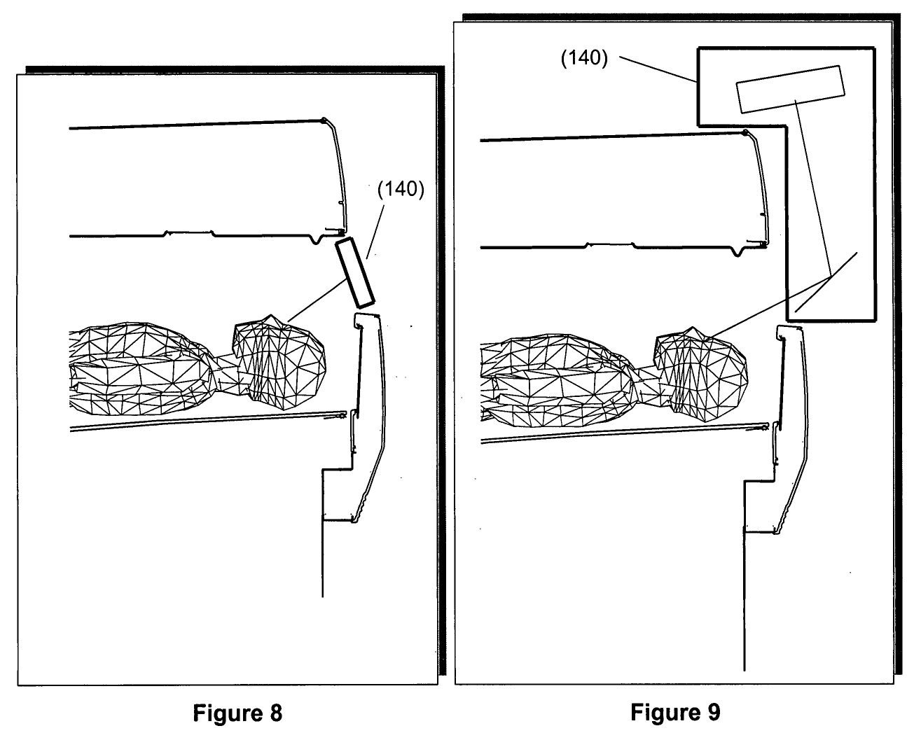

FIG. 8 and FIG. 9 show a portion of a browning bed according to the invention, in which assemblies, being different to FIG. 6, of the arrangement for emitting optical signals are shown.

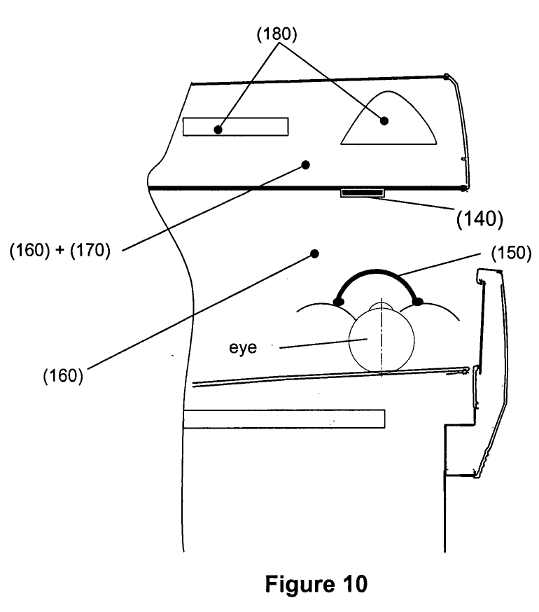

FIG. 10 schematically shows an embodiment of a browning bed according to the invention, in which sub-fractions of the radiation emitted from the radiation sources are screened by the device according to the invention, while other portions of the emitted radiation are screened by the apparatus.

The device according to the invention for protecting the eyes of a person or user against radiation is, in its most general embodiment, a radiation protection device preventing that radiation impinges the eye of the person due to the physical propagation and orientation or against which the person cannot be protected by other means when exposing towards this radiation, if so (even with closed eye lids), enters the eye and has harmful effects there. The effects can be temporarily (and therefore possibly not permanently affective), occurring for example then, when looking into bright (visible) light—especially light with a wavelength in the range of 400 to 550 nm—with open eyes and subsequently having a bright spot in the field of vision preventing for a certain time that the eye/the eyes properly realise normal optical signals. The effects can be permanent (in particular permanent affecting), for example, when over a longer time UV-radiation impinges the eye/the eyes irreversibly harming the optical nerve and/or the retina. According to a preferred embodiment, however not limiting embodiment of the invention, the invention relates to a device for protecting the human eyes from UV-radiation and/or intensive visible radiation. Further, the invention preferably relates to a device for protecting the human eyes from natural UV-radiation and natural visible radiation occurring, for example, in natural environment in sunshine, or alternatively, relates to radiation occurring in an environment with artificial UV-radiation and/or artificial radiation.

An example for natural UV-radiation and/or natural visible radiation is the radiation in the mountains or at the sea, in particular then, when the reflection of the radiation is performed at present media, for example snow or water, or also the radiation at artificial waters. It is known that for example intensive radiation at snow in the mountains leads to a phenomenon which is denoted with “snow blindness”, when the eyes are intensively contacted with the predominant UV-radiation deriving from the solar radiation and/or intensive radiation in the visible range.

An example for an artificial UV-radiation and/or artificial visible radiation, with which the invention is exemplary elucidated, however, without being limited to this embodiment, is the artificial UV-radiation and/or artificial visible radiation occurring in a tanning salon during browning. In the latter case, a person or user exposes a part of the body, be it only the face, be it another part of the body like the extremities (arms, legs, however, in particular preferred the whole body, to a radiation for browning appropriate for that purpose in a browning apparatus appropriate therefore. The radiation emitted from UV-radiation sources, for example UV-radiator tubes or high pressure burners comprise partially at least a UV-component effective for browning, even, when it does not substantially consist of a UV-component, with the fraction being effective for browning. In the cases, when browning is performed in an browning apparatus including a browning of the face, conventionally, the radiation is emitted from UV-radiator tubes or high pressure burners—as known from prior art. The spectral composition of the radiation is substantially appointed by the substances contained in the tube(s) or in the burner, conventionally phosphor, wherein the intensity of the radiation can be controlled by the apparatus, what is known by the technical expert in this field and which is not needed to be further elucidated here. For this reason the spectral composition of the UV-radiation can be controlled such that no UV-components are contained harming the skin exposed to the radiation.

In the following description reference is made to FIG. 1. For protection of the eyes against radiation comprising at least a fraction of UV-radiation and intensive visible radiation, one eye or the eyes of a person and user, respectively, are covered with the device 1 according to the invention. This comprises at least an arrangement 3 which is at least partial little translucent for covering one eye or both eyes of a person or user, and an arrangement 7 for attaching the device 1 to the head of the person and the user, respectively, who's eye(s) should be protected. The cover of the eye and the eyes, respectively, means, within the scope of the present invention, that the arrangement 3 should have such a size that at least the region of the face of the person or user of device 1, respectively, in which the eyes are situated, should have an all-around covering protection against the radiation. In a preferred embodiment of the invention the arrangement 3 lies tightly closed at the edges of the eye sockets of the person/user, so that no harmful light can pass the edges of the device 3 and subsequently enter the eye. This can be achieved by any manner known by the technical expert, for example by a tight fitting ring or pad.

The device 3 according to the invention can be constructed such that a device 3 has a size covering simultaneously both eyes of a person and user, respectively. This is a conceivable embodiment comprised by the invention, however, according to the invention, a less preferred embodiment, because also the region between—the eye sockets is covered. In that case that the device is used in a browning apparatus for browning, therefore, the region between the eyes is also covered and is, as a consequence, not browned. In this (less preferred) embodiment, this can be prevented such that, for example, the device 1 comprises an arrangement at the region between both eyes which is partial translucent for browning radiation, also consists of, for example, a material partial translucent for UV-radiation, whereas the region covering the eyes consists of a material which is substantially little translucent for UV-radiation and/or visible radiation or even non-translucent.

In other embodiments preferred according to the invention, one of which is shown in FIGS. 1 to 3, the device 1 is constructed such that for each eye socket a separated arrangement 3 is provided. This is advantageous because of higher flexibility. Further, the above described disadvantage to cover the region between the eyes during browning is not present with the existing by embodiment.

The arrangement(s) 3 covering the eyes can have each arbitrary form. They can be flat (for example in form of a strip) or provided with a contour, in a particular, they can have the form of extending bodies (of round or oval cylinders or hemispheres or hemispherical ellipsoid with a missing front side) for providing space for the eye lids/eye lashes for opening and closing. Another alternative form is a flat form, for example corresponding to a ball shell segment, comprising a central aperture on which a one side opened extending body is put providing space for the eye lids/eye lashes for the opening and closing, respectively. The latter embodiment is shown in FIG. 3, for example. Basically, the above-mentioned forms or examples are not limiting the invention; the technical expert is not limited to any form of arrangements 3 as long as they allow reliable covering of the eyes and provide a desired protection of the person/user against radiation.

Under the term “little translucent for radiation” within the scope of the present invention, it is understood that the material of the devise(s) 3 is adapted such that the amount of radiation passing through the material is such low that damages of the eyes have not to be suspected. Basically, for the protection of the eyes against harmful radiation in general and against harmful radiation in UV-range and/or in visible range, there are technical bench marks or standards; the material has to cope with the technical bench marks or standards anyway. At least, “little translucent for radiation” means that the material or the materials of which the device(s) consist of, comprise(s) such a little transmittance in general, in particular preferred according to the invention for UV-radiation and/or a visible radiation such that the bench marks or maximum values serving for protection are met. On the other hand, the invention comprises also materials for device(s) 3, constructed such that they are completely or substantially non-tanslucent for radiation, according to the invention, especially preferred for UV-radiation and/or visible radiation. Intermediate values between the mentioned extreme values are possible. The device(s) can consist of material or can consist of several materials or a composite of several materials. They can be different with respect their characteristics, in particular with respect to their respective transmittance for radiation, according to the invention especially preferred for UV-radiation and/or visible radiation, and can, if so, be synergistically supplementary.

The technical expert knows the values of the transmittance of various materials being appropriate for the device(s) 3 or can detect them without any major experimental efforts.

Especially preferred is/are according to the invention one or several material(s) having a transmittance for UV-radiation with a wavelength of λ≦400 nm, namely for UV-radiation in the range of 400 nm≧λ>320 nm, at most at 1%, related to the emitting UV-radiation and the transmittance thereof, for UV-radiation with wavelengths λ≦320 nm, namely for UV-radiation within the range of 320 nm≧λ≧250 nm, at most at 0,1%, related to the entering UV-radiation. In particular, the preferred transmittance of the material(s) of the device(s) 3 for UV-radiation is nearly at 0 (zero), related to the emitting UV-radiation. The transmittance of the material(s) used for the device(s) 3, according to the invention, for visible radiation (e.g. radiation with a wavelength λ within the range of λ≧400 nm, in particular with wavelengths of 400 nm≦λ≦550 nm) lies at: most at 5%, related to the entering visible radiation. Also, in this case of the visible radiation, the transmittance is possibly low, preferred at <5% of the entering visible radiation. In this case that the device(s) 3 consist(s) of a material, the one material impinges the above-mentioned specification of the transmittance of the UV-radiation and/or visible radiation. It is natural that thereby the standards relevant for the user have to be met. In the case that the device(s) 3 consist of several materials, the several materials impinge together or one of the several materials separately impinges the above-mentioned specification of the transmittance for UV-radiation and/or visible radiation. These embodiments of the invention comprise also a material or several materials, on which layers for reducing the transmittance for radiation, in particular for UV-radiation and/or visible radiation is attached by procedures known by the technical expert, for example sputtered or evaporated. Also the attached materials can be attached as separate materials or as mixtures or composites of such materials, for example sputtered or evaporated.

The material for the devise(s) 3 can be each known material, as long as it impinges the above-mentioned specification or a lower transmittance of radiation. According to the invention, materials which can be easily manufactured in mass production are preferred, for example thermoplastic or thermosetting plastic or glass materials or mixtures or deposit materials thereof. It is also conceivable within the region of device 1 covering the eye and the eyes, respectively, of the person and the user, respectively, to use one material or several materials substantially being little translucent or non-translucent for radiation, in particular for UV-radiation, while the regions of device 1 not used for covering an eye or both eyes, respectively, of the person and the user, respectively, could consist of another material or several other materials, which transmittance of radiation, in particular of UV-radiation, is higher or is substantially translucent for radiation, in particular for UV-radiation.

According to the invention, the device 1 comprises an arrangement 7 for attaching the device 1 to the head of the person and the user, respectively. The attachment arrangement can be a simple elastic tape put around the head or an ear/both ears of the person and the user, respectively, or can be a rigid arrangement 7 routed around the (back) head or the ear/the ears of the person and the user, respectively, and to the ends thereof arranged in the face region the arrangement 3 and (in case of an arrangement 3 for each eye socket) the both arrangements 3, respectively, is attached. The technical expert knows such arrangements in several forms of appearances and embodiments such that he can use them for device 1 of the present invention implicitly. Preferably the attachment arrangement 7 is an elastic tape.

In a further preferred embodiment, in particular in this case, in which the attachment arrangement 7 is an elastic tape, the device 1 comprises also an arrangement 5 variably or rigid connecting the both arrangements 3 within the face region of the person and the user, respectively. Arrangement 5 serves to ensure a reliable fit of the whole device 1, in particular a fit providing a reliable protection against radiation with arrangement 3 located in front of the eyes of the person and the user, respectively. Moreover, a variable arrangement 5 allows adapting the size of the whole device 1 to different anatomic conditions of the user. In the in particular preferred embodiment, also arrangement 5 is an elastic or a rigid tape, with particular advantage of an elastic tape.

According the invention, the device according to the invention comprises one or several device(s) 9 enabling a receipt of optical signals from a location being spaced from the device 1. Within the scope of the present invention, it has to be understood that from a location (it is conceivable also from several locations) being arranged in more or less larger distance from the device 1, optical signals can be sent or forwarded to the device, wherein the optical signals are received by device 1 and are presented to the person wearing the device 1 and user, respectively, for vision. The distance can be arbitrary large in that case in which the receipt of optical signals is wireless. For example, a transmitter/receiver assembly is conceivable, at which the transmitter is located at an arbitrary location and the device 1 is provided with an appropriate receiver. Alternatively thereto, the receiver can be available near the device (for example a static receiver in direct surrounding of device 1 or a mobile (for example portable) receiver in direct surrounding of device 1), and in a preferred embodiment such a receiver can be connected with a cable connection or a similar manner known by the technical expert with the device 1 for transmitting the optical signals or for transmitting electrical or electronical signals and the conversion thereof in optical signals being made visible to the eye/the eyes of the person and user of the device, respectively.

Also covered by the invention is a preferred embodiment according to which on the optical signals are made visible such that the arrangement(s) 3 of device 1 can be its/their inner side, facing the eye/the eyes of the person and the user, respectively, when wearing the device 1 at least a display, for example a LCD, which can be a micro display or at least a layer or at least a film, which allows the replay of optical signals in a recognisable form of the protected eye or the protected eyes, respectively, in particular in a visible form. In particular preferred is a LCD-911. The arrangement(s), for example display(s), layer(s), film(s) at the inner side of the respective arrangement 3 of device 1 is/are controlled by a receiver directly arranged at the device or via a cable connection, and the received signals are transferred into optical signals in a manner known by the technical expert, wherein the optical signals become visible on the film 911, and therefore recognisable for the eye/the eyes. In that way, the transmission of signals in optical manner recognisable for the eye from a location spaced more or less form device 1 or even from several such locations to device 1 is possible. The distance can be a few centimeters up to several hundred of kilometres without limiting the invention there through.

According to further preferred embodiments of the invention, the optical signals are received and/or made visible such that on the inner side of the arrangement(s) 3 a plurality of small (if necessary movable) mirrors is provided, generating optical micro arrays, which can be recognised by the eye protected form harmful radiation, and, therefor, can be reviewed.

The substantial advantage of the above-mentioned embodiments is that the receipt of optical signals from the transmitter is substantially independent and therefore complete mobility of the person/user of device 1 is ensured. Further, the type of received optical signals is not limited to special transmitters, but allows substantially over all the full bandwidth of the receipt. For example (without limiting the invention hereby) during hiking in the mountains, a hiker can protect his/her eyes from natural UV-radiation and visible radiation by the device 1 formed as spectacles according to the invention and, simultaneously, can receive optical signals from a location far away, for example entertainment-signals or fact-signals, but also information about changing weather conditions and/or path-information in form of a hiking map or information about flora and fauna of the region in which the user is situated at present, or similar. In the latter embodiments, in which the information is fact orientated, for example a coupling with a known GPS-system can be provided for making the device sensible, where the user is located at present for adapting the fact-information to the location as soon as it is required.

Another in particular preferred embodiment of the device 1 according to the invention serves for—which has been mentioned above—protection of the eyes of a person and user, respectively, against artificial radiation, being it artificial UV-radiation and/or artificial visible radiation. In further preferred embodiments, such UV-radiation and/or visible radiation is originated by apparatuses generating such radiation, for example welding apparatuses, arc generating apparatuses or—according to in particular preferred embodiments of the invention—from apparatuses for browning the skin of a person and user, respectively. Such browning apparatus—as known by the technical expert—are provided with radiation sources, for example UV-radiation sources, which emit for example radiation within the UV-range and/or visible range. Examples for such radiation sources are tubes or high pressure burners. Radiation emitted from such radiation source during operations browns the skin in gentle and harmless manner, when it is properly applied, however, is at least uncomfortable for the unprotected eye (visible radiation) or even harmful (UV-radiation).

Due to this danger, during browning, the person and the user, respectively, should wear a protection for the eyes against UV-radiation, appropriately also against the intensive radiation within the visible range. Therefore, in particular preferred embodiments of the invention, the device 1 comprise also one or several arrangement(s) allowing a checking, if the person or user of a browning apparatus, respectively, is properly wearing a device 1 for protection of the eyes against UV-radiation and/or radiation within the visible range, e.g. is wearing such that the desired goal, namely the protection of the eyes against the UV-radiation and/or the visible radiation, is achieved. With particular advantage, this is achieved that between the browning apparatus and the device 1 according to the invention a checking arrangement is switched which is chosen from electrical checking arrangement, electronical checking arrangement, radio controlled checking arrangement and a combination or several of such arrangements. Basically, such arrangements are known by the technical expert and any detailed elucidation is not necessary here. Such checking arrangements commonly function such that the browning apparatus is “messaged” in one of the described ways, if the person and the user, respectively, properly wears the device 1, e.g. such that his/her eyes are protected against UV-radiation and/or visible radiation corresponding to the common specification, for example, according to the specification described in connection with the arrangement 3, the transmittance of UV-radiation and/or visible radiation. In the following, this is further elucidated in detail. In case the specified protection of the eyes is not ensured, the browning apparatus is not switched on, i.e. a browning procedure cannot start. When the protection is not continued, for example, since the person/user takes off the device, the browning apparatus is switched off, i.e., the browning procedure is interrupted.

Also within the scope of the usage of the device 1, according to the invention, during browning in a browning apparatus the transmission of optical signals to the device 1 can be performed according to the above-mentioned case: Optical signals or electrical or electronical signals transformed into optical signals can be transmitted from a location spaced more or less far from the device 1 to the device 1 and are made visible therein for example by a film on the inner side of the arrangement(s) 3, i.e., on the side facing the eye/the eyes, for the eye/the eyes of the person and the user, respectively.

This is according to an in particular preferred embodiment of the invention (schematically shown in FIG. 4), since, usable with advantage, that the arrangement 9 for receiving optical signals comprises at least an optical element 93 for optical transmission of visible light signals. According to the invention, it is understood without limiting the invention, an arrangement directly emitting optical signals is arranged at a location spaced from the device 1 (and also from the browning apparatus, in which device 1 is used). The arrangement emitting optical signals can be each known by the technical expert or due to his technical knowledge conceived arrangement for emitting optical signals. For example, preferred embodiments of the inventions are displays, monitors (television, computer, hand-held cell phones) as arrangements for emitting optical signals without limiting the invention. In the preferred case of using the device 1 in connection with the browning in a browning apparatus, such an arrangement emitting optical signals is conventionally arranged out of the stay area of the person or user of the browning apparatus, respectively. However, this has not to be necessarily so. Preferred assemblies are assemblies of the arrangement for emitting optical signals at the head end of the browning apparatus or lateral to the browning apparatus, preferably also in the altitude of the head of the person and the user, respectively.

The optical signals in a further preferred embodiment are light signals within the visible range. The emitted signals are received in the device 1 comprising arrangement 9 for receiving optical signals, in particular preferred from a system comprising at least an optical element 93 for optical transmission of visible light signals and treated such that the received signals become visible for the eye.

In further preferred embodiments according to the invention the optical element 93 of the system for optical transmission of visible light signals is chosen from one or several mirror(s), a prism or several prisms and combinations of the mentioned optical elements. The mirror can be a conventional mirror without producing any optical additional effect (beyond mirroring), or can be a standard mirror with mirroring effect on the front face producing, if necessary, additional effects like magnifying and/or adapting to optical ametropia. When an alternative embodiment of a prism is used, this prism can be a reflection prism with or without additional optical effects (if necessary, like magnifying and/or adapting to ametropia). There could be used several mirrors or several prisms or a combination of a prism/prisms and a mirror/mirrors.

Therefore, in practice, the optical signal(s) are preferably emitted from the arrangement emitting the signal(s) and directly routed (i.e., without any indirect way) to the system comprising at least an optical element 93, preferably to the surface of the mirror or of several mirrors or—also preferred—to the surface of a prism or several prisms or a combination thereof. From there, the picture is mirrored or reflected to the eye and the eyes, respectively, of the person and user of device 1, respectively. It is conceivable even less preferred according to the invention—that the path of the optical signals is via several mirrors, prisms or a combination of such elements; however, a mirror or prism is preferred due to the simple construction and the true to original reflection of the light signals emitted by the source emitting visible signals. According to the invention, in particular preferred is an embodiment of the device 1 with the angle α preferably within the range from 5° to 75° towards to horizontal direction, wherein the visible light enters at the angle α from the arrangement emitting such signals.

An arrangement 1 is according to a further preferred embodiment of the invention, in which the arrangement 9 for receiving optical signals comprises an aperture in the arrangement 3 being little translucent for radiation, wherein arrangement 3 lets pass the optical signals to at least one optical element 93, which on its part—as described above in detail—routs the optical signals to the eye and the eyes, respectively, of the person and the user, respectively. Alternative thereto, it is also according to the invention preferred, when the arrangement 9 comprises a side window 97 of the surface in the arrangement 3 little translucent for radiation, wherein the side window 97 is translucent for visible light and lets pass the optical signals to at least one optical element 93, which on its part—as described above in detail—routs the optical signals to the eye and the eyes, respectively, of the person and the user, respectively. With both embodiments, it is ensured independently from each other that due to the preferred characteristic of the arrangement(s) 3, i.e. to be little translucent for radiation (preferably including visible radiation), the emitted optical signals designated for transmission to the eye and to the eyes, respectively, of the person and the user, respectively, can be routed to the eye, wherein thereby no UV-radiation and visible radiation, respectively, of the UV-radiator tubes or a portion thereof arrive on or in the eye and the eyes, respectively. In this case, when the arrangement 9 comprises a side window 97 translucent for a visible light, this side window is a window made of an arbitrary, for this purpose appropriate and for the technical expert easily selectable material from a group of known materials. Without limiting the invention, examples are glass and various plastics. Further preferred is the side window supplementary properly fit inserted in the material of the arrangement 9.

With the above described preferred embodiments of the invention, a radiation path is provided comprising only one intermediate step between the device emitting optical signals (for example a display of a computer) and the eye and the eyes, respectively, of the person and the user, respectively, without losing optical signals, for example by the material of the arrangement(s) 3, which should be little translucent for UV-radiation and —or visible radiation to protect the eye and the eyes, respectively, of the person or the user of the browning apparatus, respectively. Preferably—and desired in result—the at least one optical element (a mirror, for example), in relation to the eye of the person or the user of the device 1, respectively, is arranged in the light path of the optical signals such that only the (desired) optical signals reach the eye and the eyes, respectively, however, not the UV-radiation or a visible radiation, originating from the UV-radiator tubes. Therefore, the light path of the optical signals is desired such that it does not coincide with the light path of the UV-radiation and/or visible radiation originated from the UV-radiator tubes. Therefore, it is certainly prevented that the eye/the eyes of the person and the user, respectively, is/are in contact with the UV-radiation and/or visible radiation from the UV-radiator tubes. The technical expert can choose the light path by avoiding the entering of the UV-radiation and/or visible radiation from the UV-radiator tubes performing few simple attempts according to the basic laws of optics, and, therefore, there is no need to describe this in detail here.

Especially preferred, according to the invention, is an embodiment of the device 1 comprising a shutter 99 mounted in the arrangement 3 for screening the UV-radiation and/or visible radiation originated from the UV-radiation sources, for example UV-radiator tubes of a browning apparatus. Such an embodiment has the advantage that the shutter 99 ensures a reliable screening of the UV-radiation and/or visible radiation from the UV-radiator tubes in closed condition, and, therefore, it prevents that such radiation reaches the eye and the eyes, respectively, of the person and the user, respectively, without disturbing the vision impression originated by the visible light signals and being preferred passing to the eye. In particular, this is important when taking into consideration the fact that the light path between the device for emitting optical signals via the aperture or the side window 97 in the surface of the arrangement 3 and via the at least one optical element 93 to the eye or to the eyes of a person or user of the browning apparatus, respectively, is “closed” only then (thus, the light successfully reaches the eye), when certain angles are kept. The angle of the at least one optical element is preferred (even, not necessarily) a fix (and therefore not always free selectable) parameter for the device 1. However, since the position of the head of the person and the user, respectively, altogether wearing the device 1 is variable, the light path can be interrupted by moving the head. As a result, in such a case, UV-radiation and/or visible radiation could pass through the opening or the translucent side window 97 in the arrangement 3 and, therefore, to the at least one optical element 93, and, finally, in the eye and the eyes, respectively, of the person and the user, respectively. However, this is not desired and is successfully prevented by the shutter 99.

The shutter 99 can be every arbitrary shutter known by the technical expert of this technical expert field for the above described purpose. Especially preferred according to the invention are mechanical shutters and optical shutters, for example common and known LCD-shutters. In particular, optical shutters 99 are preferred in particular.

Further, an embodiment of the device 1 according to the invention is in particular preferred, according to which the shutter 99 is controllable and in particular controllable depending on data determined by a sensor mounted in the arrangement 9. The sensor can be an arbitrary sensor, without limiting the invention, for example a mechanical sensor, an electro-optical sensor, a photodiode, if necessary, with a filter effective in a certain range of wavelength (for example a band-pass filter in a range of 400 to 550 nm), an edge filter directed to a certain wavelength (for example an edge filter for the wavelength of 500 nm), or another arbitrary sensor.

The sensor detects data and the detected data are further directed to the controller being also comprised in the preferred embodiment. The controller controls the shutter 99 depending on the detected data.

In principle, the controlling can be performed such that in a further preferred embodiment of the invention the sensor detects the position of the device (1) with respect to the light source (for example, a mechanical sensor) and/or the light entering the arrangement 9 of a certain wavelength, for example the light from the UV-radiator tubes, or another physical parameter, for example an equivalent physical parameter of light entering the arrangement 9 (electro-optical sensor). The detected data are directed to the controller. Depending on the parameter detected by the sensor and subsequently on a comparison between the detected parameter with given values of the same parameter, the controller controls the shutter 99 and closes or opens the shutter 99 according to the result of the data comparison. The closing or opening can be a full closing or opening or a partial closing or opening. The closing or opening can be performed depending on the detected data stepless or in steps. This results in that, when controlling the shutter 99 in such manner, an amount of radiation (in particular preferred an amount of UV-radiation and/or visible radiation, especially preferred such radiation from the UV-radiator tubes) shines to the eye of the person or user of the browning apparatus, respectively, protected by the device 1, wherein the amount of radiation is at least equal or, in particular preferred, lower than the maximum of the permissible amount of radiation. In particular, further preferred is that no harmful radiation shines to the eye and the eyes, respectively, of the person or the user of the browning apparatus, respectively, when the control closes the shutter 99, for example when nodding the head of the person and the user, respectively, wherein the nodding should result in radiation shining to the eye according to the mechanical data, or when detecting UV-radiation and/or visible radiation from the UV-radiator tubes in the arrangement 9 by the sensor and transmitting respective data from the sensor and the control of the shutter 99.

The electronics for the above described embodiment, comprising the shutter 99, the sensor and the control, including software for their analysis, is known by the technical expert, and he can choose these components with his technical knowledge for the respective purposes and couple them with the described hardware. It is not necessary to describe this in detail here.

A further preferred embodiment of the device 1 according to the invention comprises the case—contrary to the above described embodiment of device 1, with both arrangements 3 covering the eyes of the person and the user, respectively, comprise one or several arrangement(s) 9 enabling the receipt of optical signals from a location spaced from the device 1—that only one of both arrangements 3 covering the eyes of the person and the user, respectively, comprise one or several arrangements 9 enabling the receipt of optical signals from a location spaced from the device 1 (monocular embodiment), while the other arrangement 3 covers only the eye of the person and the user, respectively, against harmful radiation like UV-radiation and/or visible radiation without enabling a receipt of optical signals. This embodiment, which either can be embodied such that the left eye receives the received signals via the corresponding at least one optical element or, alternatively, the right eye, while the corresponding other eye of the person and the user, respectively, does not receive signals, wherein this embodiment has the advantage according to the invention that the device 1 and in particular the optical element 93 does not require an exact optical alignment between two eyes of a person and user, respectively, as it is necessary for a solution comprising the optical element 93 for both eyes of the person and the user, respectively. Thus, the “monocular” solution can be in particular advantageous, when the distance between the arrangement emitting visible light signals and the device 1 is small, or both eyes of the person and the user, respectively, are different capable of receiving optical signals (different ability to see of the eyes). On the other hand, an advantage of a binocular solution is, as has been described above, that amblyopia of one eye of the person and the user, respectively, can be compensated by the second eye having higher vision resulting in a better detection of the optical signals for the user.

In a further preferred embodiment, schematically shown in FIG. 10, screening of the eye/the eyes of a person and user, respectively, against harmful radiation, for example UV-radiation 160 and/or a visible radiation 170, is performed not only by the device 1 according to the invention. Rather, radiation emitted from one or several radiation source(s) 180 is screened corresponding to radiation fractions of different wavelength, on the one hand, by the material of the arrangement(s) covering the eye and, on the other hand, by one or several arrangement(s) provided nearby the radiation sources. According to the invention, in a particularly preferred embodiment shielding against visible radiation can be carried out by a filter device 150 located nearby the radiation source(s), for example, attached in a browning apparatus on the rest face and the protection face, respectively, or arranged thereon with the face arranged between the radiation sources and the person and the user, respectively. The UV-radiation component (in this case desired for browning) passes though this filter device 150 and can impinge on the body. In the region of the eyes of the person and the user, respectively, the UV-radiation is, however, shielded by the device 3 being little or not translucent for UV-radiation. Hence, a reliable protection of the eyes against UV-radiation is performed. It is natural that this embodiment can comprise also a shutter mounted in the device 3 as well as the above described sensor and control devices.

According to a further also preferred embodiment of the invention, device 1 comprises arrangements allowing airing for preventing fogging of the arrangement 3 caused by humidity, sweat, or similar. Airing arrangements can be appropriate slots (preferably for preventing radiation access) allowing exchange of air with the surrounding, however, also other mechanical arrangements serving for airing. Alternatively thereto, or additionally, a face or several faces of the arrangement(s) 3, in particular such face(s) lying in the path of rays of the optical signals, can be made of a material preventing fogging or covered (for example sprayed or evaporated) with such a material.

The optical signals directed by the at least one optical element 93 of the arrangement 9 and finally to the eye/the eyes of the person and the user, respectively, can be arbitrary optical signals. In particular preferred embodiments the optical signals are chosen from television film signals, movie film signals, video film signals, CD-film signals, DVD-film signals, internet signals, music clip signals, mobile phone display signals, video game signals, game signals and paddle signals, wherein this does not limit the invention, and also other optical signals can be chosen. A control of the signals (for example the choice of a program, the change from one program to another, interactive games, etc.) can be controlled in a known manner, for example with a combination of mechanical and electrical pulse activators, a so called joy stick or similar known arrangements. Such an arrangement, if necessary, can be coupled or spherically connected with safety arrangements, for example with a bottom for switching on or off the arrangement in connection with the browning process. Such arrangements do not limit the invention.

Further, the present invention relates also to a browning apparatus 100. The browning apparatus 100 according to the invention comprises the following components:
(a) a bed 110 with a rest face 112 basically transparent for browning radiation for a user, and radiation sources 114 emitting browning radiation;
(b) a movable top part 120 with a protection face 122 substantially transparent for browning radiation, and UV-radiation sources 124 emitting browning radiation;
(c) wherein the top part 120 can be opened in relation to the bed 110 for entering of a user and can be closed at the beginning of the browning process; and
(d) control apparatuses 130 operable by the user for controlling the operation of the browning process.

According to the invention, the browning apparatus further comprises
(e) a device 1 for protection of the eyes of a user from radiation as being suggested by the invention, wherein the device 1 comprises an arrangement 3 being at least partial little translucent for the radiation for covering an eye/both eyes of the user and an arrangement 7 for attaching the device 1 to the head of the user, wherein the device 1 further comprises:
(f) one ore more device(s) 9 enabling a receipt of optical signals from another location spaced from the device 1.

The browning apparatus according to the invention can be each arbitrary common browning apparatus as being known by technical experts of the present field, with the exception of the device 1 according to the invention for protecting the eyes of a user against radiation, in particular UV-radiation and/or visible radiation, further preferred against radiation being emitted from UV-radiator tubes of the browning apparatus.

Such a browning apparatus can either brown the whole body or several parts thereof, for example the face, the arms, the legs and/or the corpus of the user. Further, browning apparatuses according to the invention comprise common browning apparatuses proceeding the browning process while lying therein/thereon, as well as such browning apparatuses in which the browning process is proceeded while sitting, standing, or else. Hence, the term “bed” does not limit the browning apparatus to a browning process proceeded while lying, rather, should also comprise, for example, the browning process proceeded while standing, and then stands for radiation sources arranged in the back and/or laterally and/or in the front region of the user.

In the preferred embodiment the browning apparatus 110 further comprises
(g) one or several arrangements allowing the checking by the controlling apparatuses 130 of the browning apparatus, whether the user wears the device 1.

This can be realised in an arbitrary manner not limiting the invention. For example, the arrangements can perform the checking via the possibility switching on the browning apparatus 110 only when the user is properly wearing the device 1. Alternatively or simultaneously, the checking can consist in that the browning apparatus 110 is switched off when the user takes off the device 1, for example takes off the device before the browning apparatus is switched off due to initiative of the user or operation of the corresponding control apparatus 130.

In a further in particular preferred embodiment, because useable with advantage, the browning apparatus 100 additionally comprises one or several arrangements 140 spaced from the apparatus for emitting optical signals which can be received by the device 1 and can be optically recognised by the user, hence, for example can be seen.

In particular preferred embodiments, the device 1 comprised by the browning apparatus according to the invention can comprise all features as described above in detail for the device 1 according to the invention. Due to brevity reasons, these features are not completely repeated in detail. All individual above described features of device 1 or, as well as, arbitrary combinations of the above described features, however, can be realised together with a browning apparatus 100 according to the invention and, therefore, are covered by the invention.

Further, the invention relates a method for browning at least one part of the body of a person or a user. This can be the face/the head, the upper part of the body (if necessary, including the head), the extremities (arms, legs) or the whole body, wherein the latter case is preferred according to the invention.

According to the invention, the browning is performed by means of arrangements for emitting radiation comprising browning radiation, as being basically known by the technical expert of this field and the user. The radiation is generated in the natural area by the sun, in the artificial area regularly—without limiting the invention thereby—by UV-radiation sources like UV-radiator tubes or high pressure burners, and radiated (filtered or unfiltered; latter is preferred) to the body of the person and the user, respectively. Therefore, for irradiation with artificial browning UV-radiation, browning apparatuses are preferably used chosen from whole body browning apparatuses, face browning apparatuses and browning apparatuses for the extremities of a person or user.

According to the invention, the browning is carried out, according to the method suggested here, under protecting the eye/the eyes of the person or user against components of the radiation being harmful for the eyes, simultaneously, under receiving optical signals with a device as being described above by means of preferred embodiments from a location being spaced from device 1.

The method according the invention can be a method for irradiation the skin for medical purposes applied due to UV-radiation deficiency disease or diseases, for healing or abatement thereof an UV-irradiation of the skin is beneficial (for example psoriasis). Alternatively or simultaneously, the method according to the invention can be a method for irradiation of the skin for cosmetic purposes, for example for pre-burning the skin before expected intensive sun irradiation (for example before a travel in areas with higher sun irradiation compared to the familiar location) or for browning the skin due to desired optical reasons. The desired purposes are also not limiting for the invention. Rather, the browning method according to the invention can be performed for each arbitrary purpose.

1. Device (1) for protecting the eyes of a person or user against radiation, comprising at least one arrangement (3) being at least partial little translucent for the radiation for covering one eye or both eyes of the person or user, and an arrangement (7) for attaching the device (1) to the head of a person or user, wherein the device (1) further comprises:
(a) one or several arrangement(s) (9) allowing a receipt of optical signals from a location being spaced from the device (1).

2. Device (1) according to claim 1, additionally comprising an arrangement (5) variably connecting both arrangements (3) in the face region.

3. Device (1) according to any of claims 1 or 2 for protecting the eyes of a person or user from natural radiation.

4. Device (1) according to any of claims 1 or 2 for protecting the eyes of a person or user from artificial radiation, preferred from radiation in a browning apparatus, in particular in a browning apparatus with UV-radiation sources.

5. Device (1) according to any of claims 1, 2 or 4, further comprising
(b) one or several arrangements allowing checking the wearing of the device (1) by the person or user by a checking arrangement, in particular a browning apparatus.

6. Device (1) according to claim 5, wherein the arrangement(s) is/are chosen from electrical checking arrangement, electronical checking arrangement and radio controlled checking arrangement.

7. Device (1) according to any of claims 1 to 6, wherein the low transmittance is a low transmittance for UV-radiation and/or a low transmittance for visible radiation.

8. Device (1) according to claim 7, wherein the low transmittance for UV-radiation is a transmittance in the range of 250 to 320 nm of at least 0,1% of the irradiated UV-radiation and in the range of 320 to 400 nm of at least 1% of the irradiated UV-radiation.

9. Device (1) according to claim 7 or claim 8, wherein the low transmittance for radiation in the visible range is a transmittance in the range of 400 to 550 nm of at least 5% of the irradiated visible radiation.

10. Device (1) according to any of claims 1 to 9, wherein the arrangement (9) for receiving optical signals is chosen from cable connector, sender/receiver, micro displays, and one or several, if necessary, movable mirrors for forming micro arrays.

11. Device (1) according to any one of claims 1 to 10, wherein the device (9) for receiving optical signals comprises at least a display, a layer or a film on the inner face of the arrangement (3), several movable mirrors for forming micro arrays comprising reproduction of optical signals in a form visible for the protected eye.

12. Device (1) according to claim 11, wherein the arrangement (9) is a LDC (911) for receiving optical signals.

13. Device (1) according to any of claims 1 to 9, wherein the device (9) for receiving optical signals is a system comprising at least an optical element (93) for optical transmission of visible light signals.

14. Device (1) according to claim 13, wherein the optical element (93) is chosen from mirror and prism.

15. Device (1) according to claims 13 or 14, wherein the arrangement (9) comprises an aperture in the arrangement (3) being little translucent for radiation, wherein the opening allows passing the optical signals to the at least one optical element (93), which on its part further directs the optical signals to the eye.

16. Device (1) according to claims 13 or claim 14, wherein the device (9) comprises a side window (97) of the surface in the arrangement (3) being little reluctant for radiation, wherein the side window (97) is reluctant for visible light and allows to pass the optical signals to the at least one optical element (93), which on its part further directs the optical signals to the eye.

17. Device (1) according to any of claims 1 to 9 and 13 to 16, wherein the optical signals originate from a monitor or display emitting optical signals, which are located outside the stay area of the person or user undergoing the browning procedure at or in a browning apparatus.

18. Device (1) according to any of claims 1 to 9 and 13 to 17, wherein the at least one optical element (93) is arranged in relation to the eye of the user in the light path of the optical signals such that substantially only the optical signals reach the eye, substantially, however, not UV-radiation or a visible radiation, originating from the UV-radiation sources.

19. Device (1) according to claim 18, wherein UV-radiation and visible radiation originating from the UV-radiation sources are shielded by a shutter (99) mounted in the arrangement (3).

20. Device (1) according to claim 19, wherein the shutter (99) is chosen from optical shutter (LCD-shutter) and mechanical shutter.

21. Device (1) according to any of claims 19 and 20, wherein the shutter (99) is controllable and controlled depending on data detected by a sensor.

22. Device (1) according to claim 21, wherein the sensor detects the location of the device (1) with respect to the light source and/or the light shining in the arrangement (9) from the UV-radiation sources and/or a variable being equivalent to the light shining in the arrangement (9).

23. Device (1) according to claim 21, wherein the controlling is performed in such a manner that the eye protected by the device (1) receives such an amount of radiation, which is smaller or at least equal to the maximum allowable amount of radiation.

24. Device (1) according to any of claims 1 to 23, wherein the optical signals received by the eye are chosen from television film signals, movie film signals, video film signals, CD-film signals, DVD-film signals, internet signals, music clip signals, mobile phone display signals, video game signals, game signals and paddle signals.

25. Browning apparatus (100) comprising
(a) a bed (100) having a rest face (112) substantially transparent for browning radiation for the user, and UV-radiation sources (114) emitting browning radiation:
(b) a movable top part (120) having a protection face (122) substantially transparent for browning radiation, and UV-radiation sources (124) emitting browning radiation;
(c) wherein the top part (120) can be opened in relation to the bed (110) for the entering user, and can be closed at the beginning of the browning procedure;
(d) control apparatuses (130) operable by the user for controlling the condition of a browning procedure; and
(e) a device (1) for protection of the eyes of a user against radiation, comprising at least an arrangement (3) being at least partial little translucent for covering an eye or both eyes of the user and an arrangement (7) for attaching the device (1) to the head of the user, wherein the device (1) further comprises:
(f) an or several arrangement(s) (9) allowing a receipt optical signals form a location spaced from the device (1).

26. Browning apparatus (10) according to claim 25, additionally comprising
(g) an or several arrangements allowing the checking of wearing the device (1) by the user by the control apparatus (130) of the browning apparatus (1).

27. Browning apparatus (100) according to claim 26, wherein the arrangements perform the checking via the possibility to switch on the browning apparatus (100) not until when the user is properly wearing the device (1), and/or to switch off the browning apparatus (100) not until when the user is putting off the device (1).

28. Browning apparatus (100) according to any of claims 25 to 27, additionally comprising an or several arrangements (140) spaced from the apparatus for emitting optical signals received by the device (1) and which can be optically realised by the user.

29. Browning apparatus (100) according to any of claims 25 to 28, comprising the device (1) additionally with one of the features according to claims 4 to 9 and 13 to 24.

30. Method for browning at least of part of the body of a person or user by means of devices emitting radiation comprising browning radiation while protecting the eyes of the person or user for components of the radiation being harmful for the eyes while, simultaneously, receiving optical signals with a device according to any of claims 1 to 24 from a location spaced from the device.

31. Method according to claim 30, wherein the arrangements are natural radiation sources, preferably the sun.

32. Method according to claim 30, wherein the devices are browning apparatuses, preferably whole body browning apparatuses, face browning apparatuses or browning apparatuses for the extremities of a person or user.

33. Method according to any of claims 30 to 32, wherein the devices are browning apparatuses allowing selective browning of skin sections of a person or user which have been covered during previous browning procedures for browning radiation.

34. Method according to any of claims 30 to 33 for medical purposes.

35. Method according to any of claims 30 to 33 for cosmetical purposes.
